(19) United States
(12) Patent Application Publication
(10) Pub. No.: US 2024/0124381 A1
(43) Pub. Date: Apr. 18, 2024

(54) Renewable Bio-advantaged plasticizer generated by reductive coupling of lignin-derived aromatics

(71) Applicants: Alliance for Sustainable Energy, LLC, Golden, CO (US); Wisconsin Alumni Research Foundation, Madison, WI (US); Massachusetts Institute of Technology, Cambridge, MA (US)

(72) Inventors: Gregg Tyler BECKHAM, Golden, CO (US); Shannon Scot STAHL, Madison, WI (US); Jack TWILTON, Hayward, CA (US); Yuriy ROMÁN, Cambridge, MA (US); Caroline Bradshaw HOYT-ANDERSON, Jacksonville, FL (US); Zhiming SU, Madison, WI (US)

(21) Appl. No.: 18/485,388

(22) Filed: Oct. 12, 2023

Related U.S. Application Data
(60) Provisional application No. 63/379,217, filed on Oct. 12, 2022.

(51) Int. Cl. C07C 67/00; B01J 23/44; B01J 23/89; C07C 303/08; C08K 5/12

(52) U.S. Cl. CPC C07C 67/00; B01J 23/44; B01J 23/892; C07C 303/08; C08K 5/12

(57) ABSTRACT

Described herein are systems and methods for the generation of bio-based plasticizers from lignin from 4-hydroxybenzoic acid (H), vanillic acid (G) and syringic acid (S) obtained via oxidative depolymerization of lignin substrates. Chemical and electrochemical methods are described for catalytic reductive coupling of sulfonate derivatives of H, G and S to generate all possible homo- and cross-coupling products. Advantageously, the provided systems and methods allow for the generation of renewable plasticizers that exhibit performance advantages relative to established petroleum-based phthalate ester plasticizers.

### CROSS-REFERENCE TO RELATED APPLICATIONS

This application claims priority from U.S. Provisional Patent Application No. 63/379,217, filed on Oct. 12, 2022, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### BACKGROUND

Lignin represents the largest source of biomass-derived aromatic chemicals and is an ideal supplement or alternative to petroleum-based feedstocks. Significant progress has been made in lignin depolymerization into aromatic monomers, but methods for conversion of lignin-derived monomers (LDMs) into value-added chemicals are still in the nascent stages of development. In connection with efforts focused on oxidative lignin depolymerization, we recognized that some of the most common products, 4-hydroxybenzoic acid (H), vanillic acid (G) and syringic acid (S), could serve as precursors to biaryl dicarboxylates. The parent analog, biphenyl-4,4′-dicarboxylic acid (BPDA), has been the focus of commercial interest as a monomer for polyesters and as the core structure for non-phthalate plasticizers for poly(vinyl chloride) (PVC). Existing methods for the synthesis of BPDA use petroleum-based precursors in multi-step routes (e.g., involving oxidative coupling, alkylation, and/or dehydrogenation steps, paired with autoxidation of alkyl groups into carboxylic acids), and they often afford a mixture of regioisomers. Reductive coupling of phenol derivatives represents a different route to BPDA derivatives that accesses a single product regioisomer. The biomass-derived H compound provides a means to access the same BPDA analog currently sourced from petroleum, while the G and S compounds that have methoxy substituents will afford BPDA derivatives that could have favorable properties (e.g., as a PVC plasticizer).

### SUMMARY

Described herein are systems and methods for the generation of bio-based plasticizers from lignin from 4-hydroxybenzoic acid (H), vanillic acid (G) and syringic acid (S) obtained via oxidative depolymerization of lignin substrates. Chemical and electrochemical methods are described for catalytic reductive coupling of sulfonate derivatives of H, G and S to generate all possible homo- and cross-coupling products. Advantageously, the provided systems and methods allow for the generation of renewable plasticizers that exhibit performance advantages relative to established petroleum-based phthalate ester plasticizers.

In an aspect, provided is a method comprising: a) providing one or more aromatic compounds selected from the group comprising 4-hydrooxybenzoic acid (H), vanillic acid (G), syringic acid (S) or a combination thereof; b) reacting the aromatic compounds with a sulfonyl chloride thereby generating one or more sulfonate intermediates; and reacting the one or more sulfonate intermediates via catalytic reduction thereby generating a coupled product.

The products generated by the systems and methods described herein may be useful as plasticizers, including for the plasticization of phthalate-based polymers. This may allow for facile recycling of consumer plastic goods or textiles. Examples of phthalate-based polymers include polyvinyl chloride (PVC) and polyethylene terephthalate (PET). Generally, plasticizers lower the glass transition temperature of a compound and/or increase the degradation temperature of a compound.

Various compounds, such as sulfonyl chlorides, may be useful in generating sulfonate intermediates which allow the aromatic compounds to become more electrophilic, allowing for facile conversion into the products described herein. Examples of sulfonyl chlorides include methyl sulfonyl chloride (CH₃SO₂Cl), toluenesulfonyl chloride (H₃CPhSO₂Cl), trifluoromethanesulfonyl chloride (CF₃SO₂Cl), or a combinations thereof.

The aromatic compounds or reactants (H, G and S) may advantageously be derived from abundant biomass materials, such as hardwood lignin, which may be present in waste streams of other common industrial processes such as paper pulping. This allows for a renewable source of reactants without relying on petrochemical based products and also may reduce costs and carbon dioxide emissions.

The provided systems and methods may produce both homo-coupled products (i.e., H—H, G-G, S—S) or cross-coupled products (i.e., H-G, H—S, G-S). Various catalytic systems, electrochemical reactants and ligands may be selected to promote specific coupled products. For H—H and G-G, Ni catalysts along with a sacrificial anode (e.g., Mg, Al, Zn) may be utilized. For S—S, NiCl₂ catalysts along with Zn chemical reductants and ligands may be implemented in catalytic reactions as described herein. Examples of useful ligands include DPEPhos and others shown in FIG. 3B. For cross-coupled products, Ni/Pd dual catalyst systems may be useful as described herein.

In an aspect, provided is a system for performing the methods described herein.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 illustrates the generation of H, G and S from lignin. Lignin is an abundant biomass-derived source of aromatics that represent potential precursors to commercially important biaryl-4,4′-dicarboxylates

FIG. 2 provides a schematic for electrochemical homo- and cross-coupling of aryl sulfonates, as described herein.

FIGS. 3A-3B illustrates Ni-catalyzed reductive homocoupling of S-OTf: translating conditions optimized with Zn reductant (FIG. 3A) to electrochemical conditions (FIG. 3B). ^{a} Yields are determined by ¹H NMR analysis of the crude reaction mixture using mesitylene as an internal standard, yields shown in parentheses are isolated. ^{b} 60° C. ^{c} DMSO solvent.

FIGS. 4A-4B illustrate Ni/Pd-catalyzed reductive cross-coupling of lignin-derived aryl sulfonates. FIG. 4A shows HTE optimization of G/S cross-coupling. Left chart: S-OTf:G-OTs=1:1; right chart: DMSO solvent, S-OTf:G-OTs=1:1.25. FIG. 4B shows optimization of electrochemical Ni/Pd-catalyzed cross-coupling. ^{a} Yields determined by UPLC-MS analysis using 1,3,5-trimethoxylbenzene as an internal standard, yields shown in parentheses are isolated. ^{b} RVC cathode. ^{c} L1=4,4′-dPhbpy, L2=dppb (3.6 mol %), DMA instead of DMSO, 60° C.

FIG. 5 provides thermal analysis of lignin-derived biaryl plasticizers. From left to right: unplasticized PVC, 10 wt % plasticized PVC with DEHP, and 10 wt % plasticized PVC with lignin-derived biaryl plasticizers.

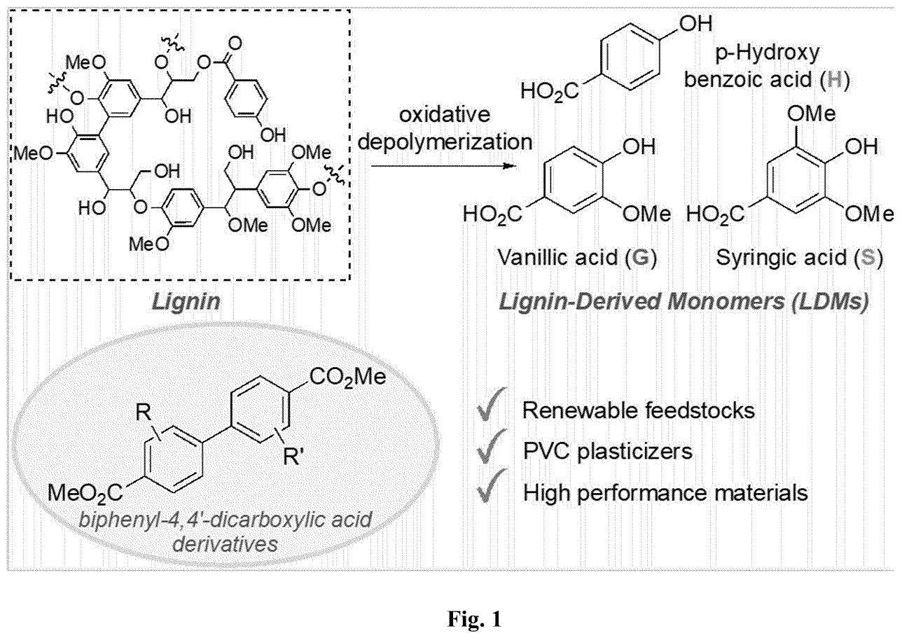

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

### Example 1—Ni- and Ni/Pd-Catalyzed Reductive Coupling of Lignin-Derived Aromatics to Access Bio-Based Plasticizers

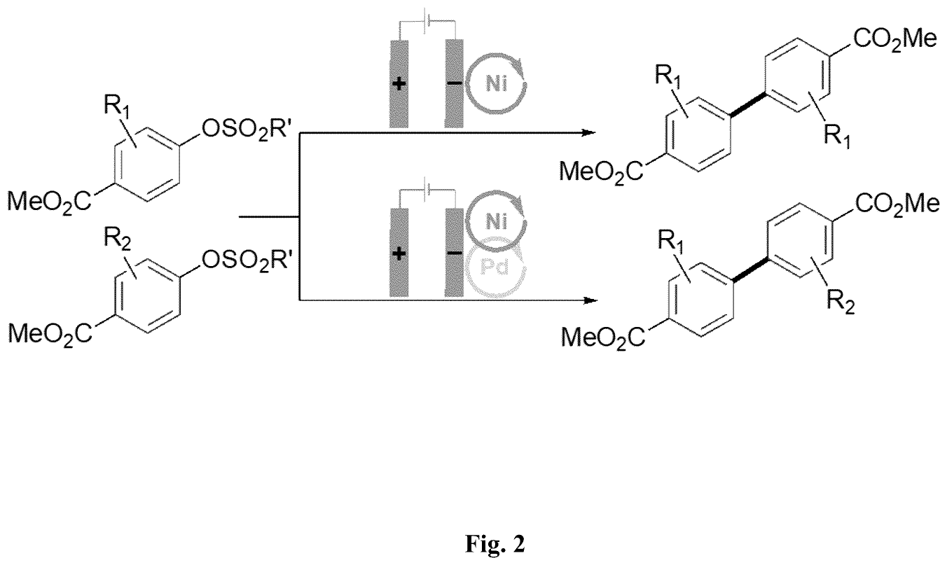

Described herein is the conversion of 4-hydroxybenzoic acid (H), vanillic acid (G) and syringic acid (S) to aryl sulfonates amenable to reductive cross-coupling. Ni-catalyzed coupling of aryl electrophiles to access biaryls were first reported in the 1970s, and the field advanced significantly in subsequent decades. These reactions typically feature stoichiometric metal reductants, such as Zn powder, but important electrochemical precedents also exist. Several examples provide an important foundation for the present work. In 1995, Percec et al. demonstrated that a Ni/PPh₃ catalyst system with Zn reductant promotes homocoupling of aryl sulfonates to biaryls. Shortly thereafter, Jutand and co-workers achieved homocoupling of aryl triflates with phosphine-ligated Pd or Ni catalysts. This study included a single example of electrochemical Ni-catalyzed homocoupling, using 1-naphthyl triflate as the substrate (Scheme 1A). In recent years, Weix and co-workers have developed methods for selective cross-coupling of aryl electrophiles with a co-catalyst system containing both Ni and Pd in the presence of Zn as the reductant. The groups of Weix and Kramer/Lian independently reported reductive cross-coupling of two different aryl sulfonates by pairing Pd/bisphosphine and Ni/diimine co-catalysts [diimine=substituted 2,2′-bipyridine (bpy) or 2,9-phenanthroline (phen)] with Zn. Methods for reductive cross-coupling of phenol derivatives are provided herein (FIG. 2).

Chemical and electrochemical conditions have complementary advantages for reductive coupling reactions. Chemical conditions are more straightforward to implement on small scale, owing to their use of standard laboratory equipment, and they are more amenable to high-throughput experimentation (HTE) techniques for catalyst discovery and reaction optimization. Electrochemical methods offer advantages for large scale applications by avoiding the challenges of handling of dense metal-powder reagents and creating opportunities to improve sustainability. Although advances have been made in the development of electrochemical reactors for parallel reaction screening, chemical HTE methodology retains substantially improved efficiency and is compatible with smaller quantities of reagents. In this context, HTE screening methods using chemical reductants could enable rapid identification of promising catalyst systems and conditions for subsequent development of electrochemical methods. The results outlined herein validate this hypothesis and achieve successful chemical and electrochemical conditions for all possible homo- and cross-coupling permutations between H-, G-, and S-derived reaction partners. Additional important outcomes of this study include (a) identification of mono- and bidentate phosphine ligands that lack precedent in Ni-catalyzed reductive coupling reactions, (b) successful adaptation of catalysts from chemical to electrochemical conditions, with matching or superior performance, (c) the first demonstration of Ni/Pd co-catalyzed reductive biaryl cross-coupling under electrochemical conditions, and (d) data showing that biaryl dicarboxylic esters prepared from LDMs exhibit improved PVC plasticizer performance and reduced toxicity relative to a commercial phthalate-based plasticizer.

### Results and Discussion

Ni-Catalyzed Homocoupling of LDMs. The methyl esters of H, G, and S are readily converted into electrophiles by reaction of the phenols with sulfonyl chlorides, RSO₂Cl [R=methyl (Ms), tosyl (Ts), and trifluoromethyl (Tf)]. Initial studies evaluated the electrochemical homocoupling of methyl 3-methoxy-4-((methylsulfonyl)oxy)benzoate (G-OMs). The two possible byproducts are denoted as the Ar—H and ArO—H species, derived from reductive cleavage of the C—O or the S—O bond of the G-OMs substrate. A combination of NiCl₂(dme)/bpy has been used previously for reductive homocoupling of Ar—X species and this catalyst system was tested initially in an undivided cell with LiBr as the electrolyte and stainless steel as the anode. However, these conditions only afforded the G-G product in 29% yield, with a significant amount of byproduct and unreacted starting material (Table 1, entry 1). Use of increased bpy ligand loading (bpy:Ni=3:1) stabilizes the catalyst and leads to a higher yield of the desired product (72%), together with the Ar—H byproduct (27%; Table 1, entry 2). Other sacrificial anodes were tested in an effort to optimize the yield of biaryl product (Table 1, entries 3-5). Significant reductive C—O cleavage was also observed when Al or Zn was used as the anode (Table 1, entries 3 and 4). This C—O cleavage is rationalized by previous observations that aryl-Ni species can transfer an aryl group to Zn²⁺, generating aryl-Zn species that are susceptible to protonolysis and Ar—H byproduct formation. Electrolysis in an undivided cell using a Mg anode proved ineffective (Table 1, entry 5). In this case, reductive S—O bond cleavage was favored, likely reflecting single-electron reduction of the sulfonyl group at the Mg surface. These considerations prompted us to test a sacrificial anode with a divided cell configuration that would avoid the contact of substrate with the anode surface and minimize the presence of Lewis acidic metal ions in the cathodic chamber. This hypothesis was validated by observation of a 92% G-G product yield when using a Mg anode in a divided cell (Table 1, entry 7). This outcome is noteworthy because it is significantly better than that achieved when performing the same reaction under separately optimized chemical conditions with Zn as a stoichiometric reductant (cf. optimal G-G yield=59%). Use of analogous conditions with H-OMs as the substrate leads to near-quantitative yield of the biaryl H—H product (Table 1, entry 8). This outcome was achieved, even when lowering the Ni catalyst loading to 1 mol %. Use of a stainless-steel anode in an undivided cell retained good yield (Table 1, entry 9). The latter conditions are readily implemented in a recirculating flow electrolysis cell with a parallel-plate reactor. This approach was used to conduct a larger scale reaction (11 g, 48 mmol H-OMs), accessing the H—H product in 80% yield with 2 mol % Ni catalyst.

TABLE 1
Optimization of electrochemical Ni-catalyzed reductive homocoupling

| | | | | Ar—H | ArO—H | Yield |
|---|---|---|---|---|---|---|
| Entry | Ar—X | Cell type | Anode | (%) | (%) | (%) |
| 1^{b} | G | undivided | stainless- | 42 | 0 | 29 |
| | | | steel | | | |
| 2 | | undivided | stainless- | 27 | 0 | 72 |
| | | | steel | | | |
| 3 | | undivided | Al | 20 | 0 | 20 |
| 4 | | undivided | Zn | 95 | 2 | 3 |
| 5 | | undivided | Mg | 7 | 37 | 4 |
| 6 | | divided | stainless- | 7 | 4 | 80 |
| | | | steel | | | |
| 7 | | divided | Mg | 8 | 0 | 92 (90) |
| 8^{c} | H | divided | Mg | 0 | 0 | 99 (97) |
| 9^{c} | | undivided | stainless- | 10 | 0 | 89 |
| | | | steel | | | |
| 10 | S | divided | Mg | 2 | 1 | 3 |

Reaction conditions: Vₐₚₚ = −1.7 V (vs Fc/Fc⁺); Cathode: [LiBr (1 equiv); DMF (0.4M), rt]; Anode: [NiCl₂ (dme)/bpy (5/15 mol %); LiBr (1 equiv); DMF (0.4M), rt].
Yields are determined by ¹H NMR analysis of the crude reaction mixture using mesitylene as an internal standard, yields shown in parentheses are isolated.
^{b}5 mol % bpy. The rest of the mass corresponds to recovered starting material.
^{c}1 mol % Ni catalyst.

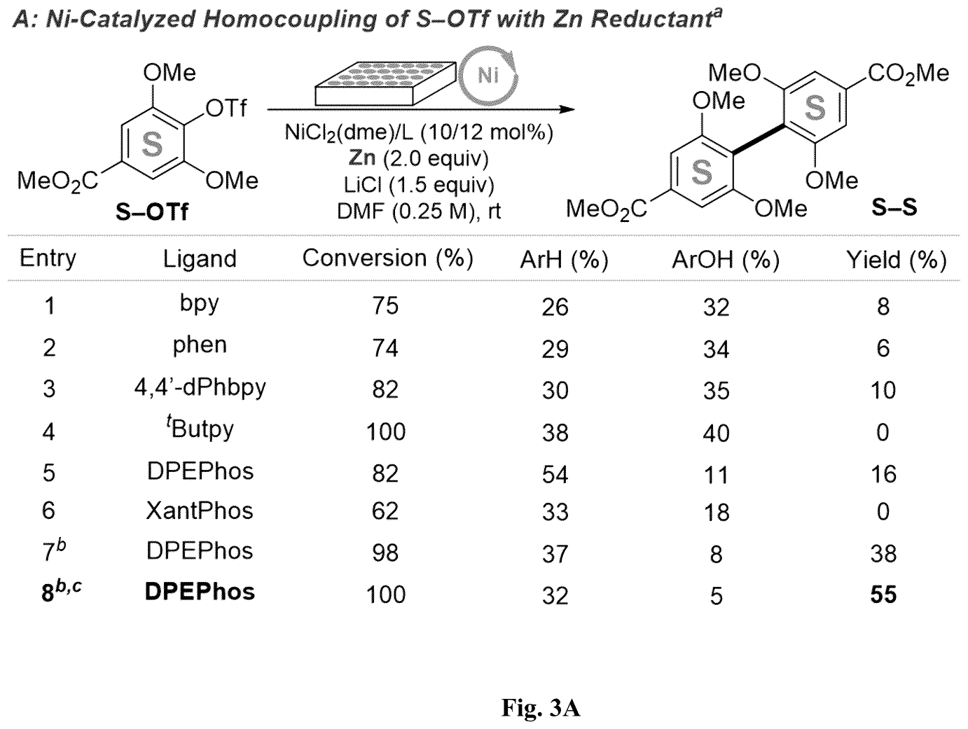

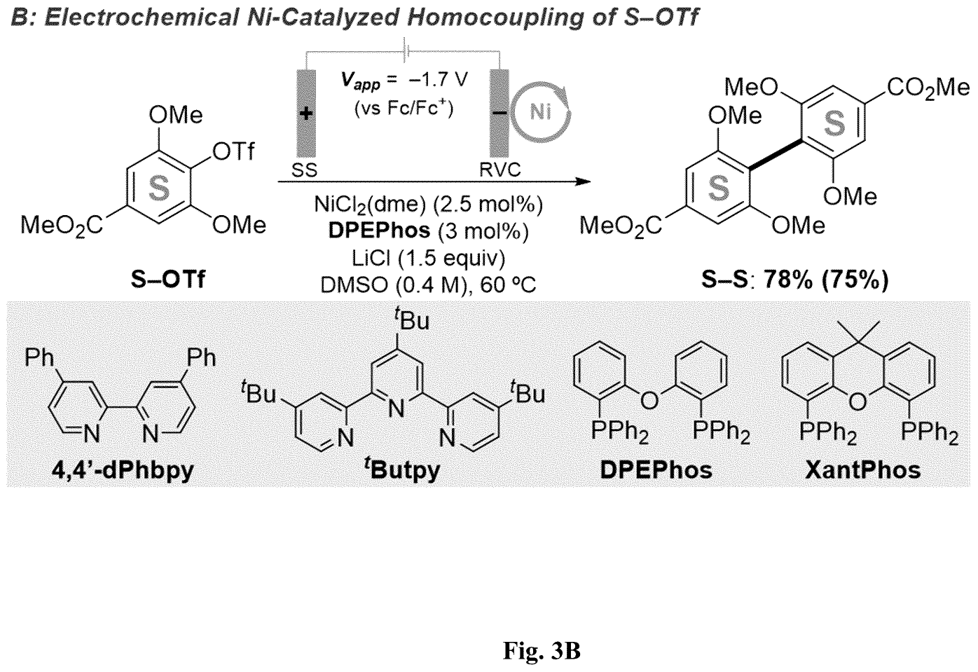

The catalyst and conditions identified for homocoupling of H-OMs and G-OMs proved ineffective with the more sterically demanding syringic acid derivative S-OMs. Only trace quantities of S—S product were obtained (Table 1, entry 10). To facilitate evaluation of modified conditions, we used a 24-well screening platform with Zn powder as a chemical reductant. The triflate derivative S-OTf was found to be more reactive than the mesylate, and this substrate was tested with dozens of nitrogen- and phosphine-based ligands. Selected results are summarized in FIG. 3A. DPEPhos was the only ligand that showed modest success; even the closely related, conformationally more rigid XantPhos ligand was completely ineffective (FIG. 3A, entries 5 and 6). Increasing the temperature to 60° C. led to an increase in conversion and product yield (FIG. 3A, entry 7), and changing the solvent to DMSO led to a 55% yield of S—S (FIG. 3A, entry 8). The outcome improved even further when the conditions were adapted to an undivided electrochemical cell with a stainless-steel anode: the desired dimer S—S was generated in 78% yield (FIG. 3B). This improved electrochemical outcome was achieved, even though the NiCl₂/DPEPhos catalyst loading was lowered to 2.5 mol %.

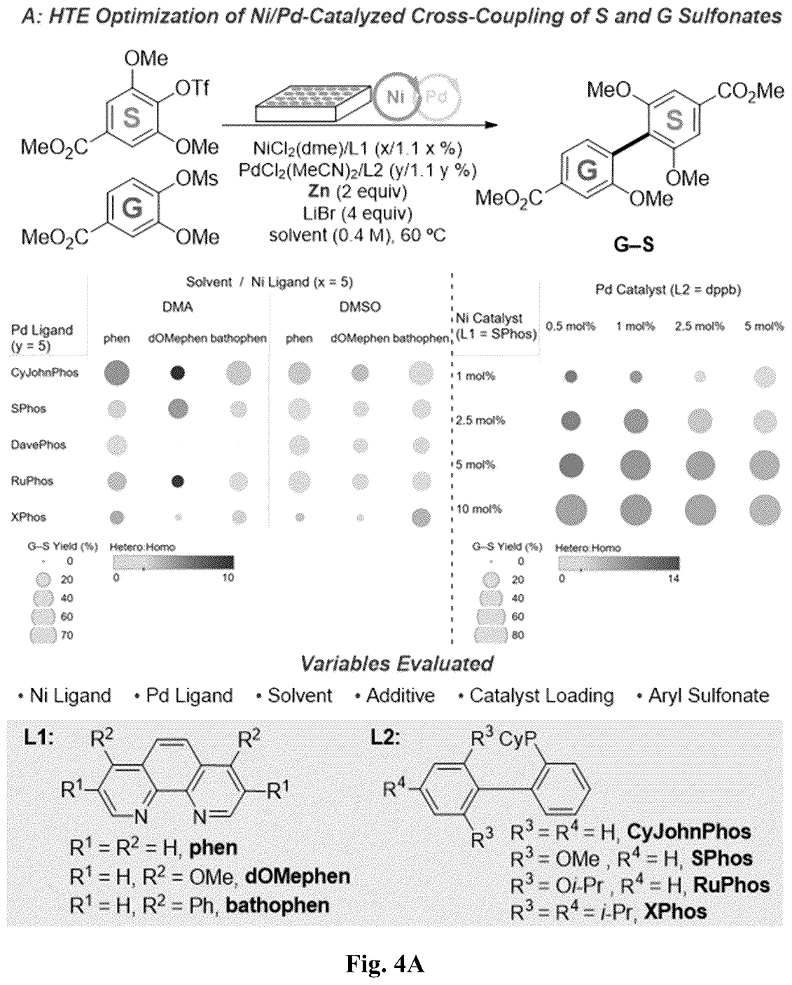

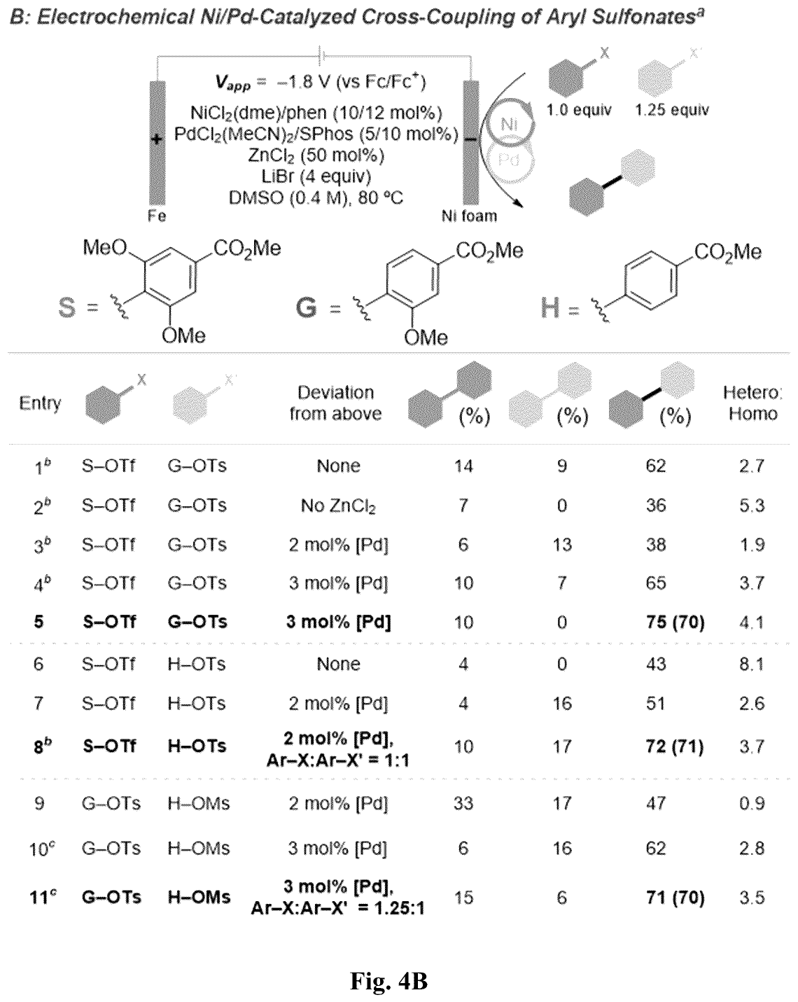

Optimization of Ni/Pd-Catalyzed Cross-Coupling. The Ni-only catalyst systems noted above were evaluated in the cross-coupling of H, G, and S sulfonates; however, these reactions led to poor selectivity and yields of the desired products. These complications prompted us to evaluate the recently disclosed dual Ni/Pd co-catalyst systems. For example, the method of Weix and coworkers, which employs Ni/Pd chloride salts in combination with 4,4′-diphenyl-bpy (4,4′-dPhbpy) and 1,4-bis(diphenylphosphino)butane (dppb) and Zn as a chemical reductant, supports cross-coupling of aryl triflates and tosylates. Efforts to translate this catalyst system to electrochemical cross-coupling of G and S sulfonates were unsuccessful, regardless of the sulfonate activating groups: biaryl products formed in ≤15% yield and favored the homocoupling products. Consequently, we again elected to use the high-throughput experimentation platform with Zn as the chemical reductant to evaluate modified conditions. Initial studies focused on cross-coupling of G and S sulfonates, evaluating different combinations of ligands, solvents, additives, sulfonate activating groups, and Ni/Pd ratios (FIG. 4A). Among the most noteworthy outcome from these experiments is the beneficial effect of bulky biaryl dialkyl monophosphine ligands (“Buchwald ligands”). The utility of these ligands could reflect their ability to promote the difficult reductive elimination steps. Overall, optimal G/S cross-coupling was achieved with a catalyst composed of 10 mol % NiCl₂(dme), 11 mol % phen, 5 mol % PdCl₂(MeCN)₂, 6 mol % SPhos.

The catalytic conditions identified with Zn as the reductant were then evaluated under electrochemical conditions (FIG. 4B). Promising performance was identified with a reticulated vitreous carbon (RVC) cathode, sacrificial iron anode, and a constant applied potential of −1.8 V vs. Fc/Fc⁺. Inclusion of 0.5 equiv ZnCl₂ significantly improved the reaction outcome (FIG. 4B, entries 1 and 2), consistent with previous evidence that Zn²⁺ salts mediate transmetallation between Ni and Pd centers. Increasing the phosphine ligand loading from 1.1 to 2 equiv with respect to Pd stabilized the Pd catalyst. These initial conditions afforded the desired product G-S in 62% yield with 23% homocoupled byproducts, similar to the outcome of the chemical screening studies with Zn as a chemical reductant. Adjusting the Ni:Pd ratio from 2:1 to 3.3:1 and using a Ni foam cathode instead of RVC increased the G-S product yield to 75% (FIG. 4B, entries 3-5). Slight modification of these conditions accessed the H—S cross-coupling product in 72% yield (FIG. 4B, entry 8). Analogous conditions were less effective for cross-coupling of the less sterically demanding H and G sulfonates (FIG. 4B, entry 9), but adaptation of the chemical catalyst system reported by Weix and coworkers proved effective for the cross-coupling of H-OMs/G-OTs, accessing H-G in 71% yield (FIG. 4B, entry 11). This reaction represents the first selective cross-coupling (under chemical or electrochemical conditions) of aryl mesylate/aryl tosylate partners, which are significantly more economical than aryl triflates

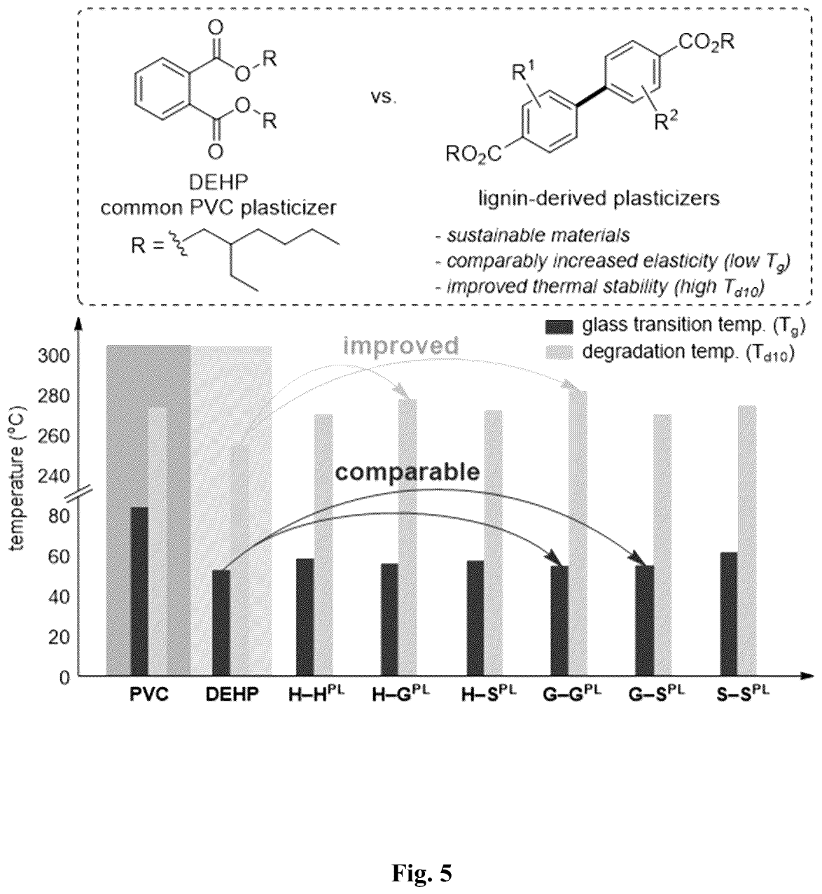

Plasticizer properties of lignin-derived biaryls. The above results provide access to all possible homo- and cross-coupled BPDA derivatives of H, G, and S. These structures provide the basis for testing of these materials as plasticizers for PVC and comparison of their performance relative to the existing petroleum-derived incumbent, di(2-ethylhexyl)phthalate (DEHP). Each of the BPDA methyl esters was subjected to Ti(OBu)₄-promoted transesterification with 2-ethylhexanol to afford the corresponding DEH-BPDA derivatives, designated H—H^{PL}, H-G^{PL}, H—S^{PL}, G-G^{PL}, G-S^{PL}, and S—S^{PL}. The thermal properties of these structures were characterized by thermogravimetric analysis (TGA) and differential scanning calorimetry (DSC). DEHP and the DEH-BPDA derivatives were then individually integrated with PVC at 10 wt %, and the composites were analyzed by TGA and DSC to identify their glass transition temperature (T_{g}) and the temperature at which the polymer degrades with 10% or 50% loss of its original weight (T_{d10}, T_{d50}). The former value reflects the ability of the plasticizer to soften PVC, while the latter values reflect the thermostability of the composite material. Preferred plasticizers will achieve lower T_{g} and higher T_{d10}/T_{d50} values. The results, summarized in FIG. 5, show that the different plasticizers lower the T_{g} of PVC from 83.0° C. to 52.1-61.0° C. The greatest effect is observed with DEHP, G-G^{PL} and G-S^{PL}, which lead to T_{g} values of 52.1, 54.4 and 54.6° C., respectively. Meanwhile, H-G^{PL} and G-G^{PL} show a notable enhancement in thermostability, with these plasticized materials exhibiting even higher T_{d10} (278 and 281° C.) than PVC itself (272° C.), and both outperform DEHP (T_{d10}=253° C.).

The results above demonstrate the utility of Ni- and Ni/Pd-catalyzed cross-electrophile coupling to convert lignin-derived aromatic compounds into a collective of substituted biphenyl dicarboxylic acids. All possible combinations of H, G, and S monomers have been prepared, with symmetrical dimers accessed using a Ni-only catalyst system and the unsymmetrical dimers accessed using Ni/Pd cocatalyst systems. The results highlight the synergy between chemical and electrochemical reduction methods. HTE screening methods with a chemical reductant offer advantages for identification of effective catalyst compositions. For example, Ni/DPEPhos and Ni/Pd-cocatalysts using sterically bulky monophosphines that lack precedent cross-electrophile biaryl coupling were identified. In each case, the chemical reaction conditions were successfully translated to electrochemical conditions, often resulting in improved performance. The beneficial effect of bulky phosphine ligands with the S-derived monomers has important implications for other cross-electrophile coupling reactions with sterically congested aryl electrophiles. Finally, the new BPDA derivatives bearing methoxy substituents, which are intrinsic to lignin-based aromatics, exhibit appealing plasticizer properties.

### CONCLUSION

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
providing one or more aromatic compounds selected from the group comprising 4-hydrooxybenzoic acid (H), vanillic acid (G), syringic acid (S) or a combination thereof;
reacting the aromatic compounds with a sulfonyl chloride thereby generating one or more sulfonate intermediates;
reacting the one or more sulfonate intermediates via catalytic reduction thereby generating a coupled product.

2. The method of claim 1, wherein the coupled product is a plasticizer.

3. The method of claim 2, wherein the plasticizer is capable of lowering a glass transition temperature of a phthalate compound.

4. The method of claim 1, wherein the one or more aromatic compounds are derived from biomass.

5. The method of claim 1, wherein the one or more aromatic compounds are derived from lignin.

6. The method of claim 1, wherein the sulfonyl chloride is selected from the group comprising methyl sulfonyl chloride (CH₃SO₂Cl), toluenesulfonyl chloride (H₃CPhSO₂Cl), trifluoromethanesulfonyl chloride (CF₃SO₂Cl), or a combination thereof.

7. The method of claim 1, wherein the coupled product is a homo-coupled product comprising H—H, G-G or a combination thereof.

8. The method of claim 7, wherein the step of reacting sulfonate intermediates is performed in the presence of a Ni catalyst.

9. The method of claim 7, wherein the step of reacting the sulfonate intermediates is performed in the presence of a sacrificial anode.

10. The method of claim 9, wherein the sacrificial anode comprises Mg.

11. The method of claim 1, wherein the coupled product is a homo-coupled product comprising S—S.

12. The method of claim 11, wherein the step of reacting the sulfonate intermediates is performed in the presence of a NiCl₂ catalyst and Zn powder and a chemical reductant.

13. The method of claim 11, wherein the step of reacting the sulfonate intermediates is performed in the presence of a nitrogen-based or phosphine-based ligand.

14. The method of claim 13, wherein the ligand is DPEPhos.

15. The method of claim 1, wherein the coupled product is a cross-coupled product comprising H-G, H—S, G-S or a combination thereof.

16. The method of claim 15, wherein the step of reacting the sulfonate intermediates is performed in the presence of a Ni/Pd dual catalyst system.

17. The method of claim 16, wherein the Ni/Pd dual catalyst system comprises NiCl₂ provided in about 10 mol % and PdCl₂ provided in about 3 mol %.
